(19) United States
(12) Patent Application Publication
(10) Pub. No.: US 2015/0259722 A1
(43) Pub. Date: Sep. 17, 2015

(54) BIOSENSOR COMPOSITIONS AND METHODS OF THEIR USE

(71) Applicant: CIRLE, Miami, FL (US)

(72) Inventor: Richard Awdeh, Miami, FL (US)

(21) Appl. No.: 14/432,322

(22) PCT Filed: Sep. 27, 2013
(86) PCT No.: PCT/US2013/062249

§ 371 Date: Mar. 30, 2015

Related U.S. Application Data
(60) Provisional application No. 61/707,211, filed on Sep. 28, 2012.
(60) Provisional application No. 61/827,302, filed on May 24, 2013.

(51) Int. Cl. C12Q 1/04; G01N 21/78

(52) U.S. Cl. CPC C12Q 1/04; G01N 21/78; G01N 2021/7786

(57) ABSTRACT

Embodiments of the present disclosure provide for biosensors that include a material such as polydiacetylene (PDA) material, where the material is used for detection of a microbe or microbial product present in a fluid present in the container. Embodiments of the present disclosure provide for containers, or structures used in conjunction with the containers, that include a polydiacetylene (PDA) material, where the PDA material is used for detection of a microbe or microbial product present in a fluid present in the container. In an embodiment, a change of PDA color (e.g., blue to red) indicates detection of the microbe or microbial product in the fluid within the container. In an embodiment, the PDA material can be selected and/or the container or structure designed so that only certain types of microbes can be detected or so that a plurality of types of microbes is detected.

### CROSS-REFERENCE TO RELATED APPLICATION

This application claims priority to U.S. provisional application entitled “Biosensor Compositions and Methods of Their Use,” having Ser. No. 61/707,211, filed on Sep. 28, 2012, which is entirely incorporated herein by reference.

This application also claims priority to U.S. provisional application entitled “Biosensor Compositions and Methods of Their Use,” having Ser. No. 61/827,302, filed on May 24, 2013, which is entirely incorporated herein by reference.

### BACKGROUND

A biosensor is an analytical device that employs biological elements such as enzymes, antibodies, nucleic acids, and microorganisms for their specific biological interactions with target items. For detection, various methods such as colorimetric detection, fluorescent detection, and electrochemical detection have been used. Colorimetric detection is the easiest and the most convenient method because detection can be done using the naked eye. Biosensors offer advantages as alternatives to conventional analytical methods because of their inherent specificity, simplicity, and quick response.

### SUMMARY

Embodiments of the present disclosure provide for biosensors that include a material, such as polydiacetylene (PDA) material, where the material is used for detection of a microbe or microbial product present in a fluid present in the container. Embodiments of the present disclosure provide for containers, or structures used in conjunction with the containers, that include a polydiacetylene (PDA) material, where the PDA material is used for detection of a microbe or microbial product present in a fluid present in the container. In an embodiment, a change of PDA color (e.g., blue to red) indicates detection of the microbe or microbial product in the fluid within the container. In an embodiment, the PDA material can be selected and/or the container or structure designed so that only certain types of microbes can be detected or so that a plurality of types of microbes is detected.

An embodiment of the present disclosure includes a biosensor, among others, including: a material, such as a polydiacetylene (PDA) material, wherein a microbe or microbial product, in a fluid contacts the material and a change of material color indicates detection of the microbe or microbial product, wherein the material is disposed on a structure of the biosensor or the biosensor. In an embodiment, the biosensor can be a container.

An embodiment of the present disclosure includes a container for detection of a microbe or microbial product in a fluid, among others, including: a polydiacetylene (PDA) material, wherein the microbe or microbial product contacts the PDA material and a change of PDA material color indicates detection of the microbe or microbial product, wherein the PDA material is disposed on a structure or on a portion of the container.

### BRIEF DESCRIPTION OF THE DRAWINGS

FIG. 1 is a schematic representation of lamellar PDA domains associated with/within a sol-gel, packaging polymer, or sol-gel packaging polymer matrix. (A. matrix, B. PDA domains, C. PDA domains associated with matrix)

FIG. 2 shows microscopy images of lamellar PDA domains on a sol-gel matrix.

FIG. 3 contains pictures showing sol-gel/PDA patches and coated plastic tubing with color changes induced by bacteria.

FIG. 4 is a schematic of the creation of packaging polymer/PDA thin sensor films at the air/water interface.

FIG. 5 is a schematic of the morphology of the packaging polymer/PDA films created at the air/water interface. (A. PDA lamellar domains, B. Polymeric matrix, C. PDA lamellar domains and polymeric matrix at the air/water interface.)

FIG. 6 is a schematic of the process in which lipid/PDA vesicles are encapsulated within a porous transparent matrix and used for microbial detection.

FIG. 7 is a schematic showing one embodiment where a PDA composition is placed adjacent to a filter.

FIG. 8 is a schematic showing one embodiment where PDA micro- or nano-islands are printed onto substrate.

FIG. 9 is a graph that illustrates the color change values (ratios of Abs₆₄₀/Abs₅₃₀) measured in glass PDA sensors vs. time at different concentrations of Pseudomonas Aeruginosa.

FIG. 10 is a graph that illustrates the % color change of glass PDA vs. time at different concentration of Pseudomonas Aeruginosa in growth medium. (Inset: Concentration of Pseudomonas Aeruginosa in growth medium at different time points).

FIG. 11 is a graph that illustrates the color change values (ratios of Abs₆₄₀/Abs₅₃₀) measured in Perspex PDA sensors vs. time at different concentrations of Pseudomonas Aeruginosa.

FIG. 12 illustrates a CR measured in Perspex PDA sensors vs. time at different concentrations of Pseudomonas Aeruginosa. (Inset: Concentration of Pseudomonas Aeruginosa in growth medium at different time points).

FIGS. 13A-13C illustrate representative examples of how points are assigned for color change.

FIG. 14 illustrates a representative example of a plate used to evaluate the biosensor.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of in chemistry, microbiology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### DEFINITIONS

As used in the specification and claims, the singular form “a”, “an” and “the” include plural references unless the context clearly dictates otherwise. For example, the term “a polymer” includes a plurality of polymers, including mixtures thereof.

“Aliphatic group” refers to a saturated or unsaturated, linear or branched hydrocarbon group and encompasses alkyl, alkenyl, and alkynyl groups, for example.

“Alkyl” refers to a monovalent group derived from a straight or branched chain saturated hydrocarbon by the removal of a single hydrogen atom. Exemplary alkyl groups include methyl, ethyl, n- and iso-propyl, cetyl, and the like.

“Alkylene” refers to a divalent group derived from a straight or branched chain saturated hydrocarbon by the removal of two hydrogen atoms. Exemplary alkylene groups include methylene, ethylene, propylene, and the like.

“Amido group” and “amide” refer to a group of formula —C(O)NY1Y2, where Y1 and Y2 are independently selected from H, alkyl, alkylene, aryl and arylalkyl.

“Amino group” and “amine” refer to a group of formula —NY3Y4, where Y3 and Y4 are independently selected from H, alkyl, alkylene, aryl, and arylalkyl.

“Amidoamine group” or “amidoamine” refer to compounds having an amine group and an amide group. “Cycloalkyl” refers to a saturated alicyclic hydrocarbon such as cyclopropane, cyclobutane, cyclopentane, cyclohexane, and the like.

The terms “diacetylene” and “diacetylene monomer” refer to a chemical having the formula of C₄H₂ (HC≡C—C≡CH).

The terms “polydiacetylene” and “PDA” refer to a composition containing two or more diacetylene monomers and having the chemical formula of I:

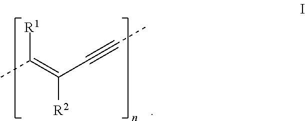

where R¹ and R2 are each independently selected from H, a C₁˜C₁₂, or C₁˜C₈, or C₁˜C₆, or C₁˜C₄ straight-chain or branched, or a C₃˜C₁₂, or C₃˜C₈, or C₃˜C₆ cyclic, substituted or unsubstituted, alkyl radical, and wherein “n” is between 1 and 10,000. The polydiacetylenes provided herein include 10,12-tricosadiynoic acid, 5,7-pentacosadiynoic acid, 10,12-pentacosadiynoic acid, 10,12-pentacosadiynoate, and 5,7-docosadiynoic acid. A “polydiacetylene solution”, a “PDA solution”, or a “PDA material” comprises a polydiacetylene as defined herein. In an embodiment, the PDA can be a fluorescent. Polydiacetylene (PDA) is widely known because of its unique optical properties. The PDA polymer is formed by the 1,4 addition of diacetylenic monomers, which is initiated by ultraviolet irradiation. The result is an intensely colored polymer, typically of a deep blue color. Among the first demonstrations of potential PDA biological applications was the colorimetric detection of the influenza virus, which relied on the reaction between the derivatized diacetylenic monomer and the cellular receptor of the virus (Charych et al., 1993. Science. 261(5121), 585-588).

The term “filter” includes any material that is capable of segregating two or more compounds. In some embodiments, a filter segregates a microbe or microbial product from a sample fluid in the sense that the filter either concentrates the microbe or microbial product in the filter or prevents the microbe or microbial product from passing through the filter. It should be understood that a filter can comprise any material including, but not limited to, cellulose, nitrocellulose, paper fibers, polyurethane, porous plastics, hydrogels, plastics or polymer films which can be made porous using gaseous or solid-phase porogens.

As used herein, the term “microbe” includes a bacterium, fungus, virus, protozoan, and yeast. Exemplary microbes include, Serratia spp., Pseudomonas spp., Staphylococcus aureus, Staphylococcus pneumonia, and fusarium (fungi). A “microbial product” includes an enzyme, peptide, lipid, or other composition secreted by a microbe. Embodiments of the present disclosure can be designed to detect a plurality of types of microbes or only specific types of microbes.

The term “packaging material” or “material to form the container” or the like are defined herein to include any material that can be used to package or contain liquids, animal products, and the like. In some embodiments, the “packaging material” comprises a plastic. A packaging material can be formed into any type of container including, but not limited to, a bottle and a bottle cap. In some embodiments, a container comprising the packaging material has a transparent window in which the PDA containing composition is placed. “Other polymeric materials” include, but are not limited to, surgical gowns, surgical dressings, contact lenses, contact lens cases, syringes, catheters, other medical consumables, and medical devices.

As used herein, the term “packaging monomer”, includes, but is not limited to, an ethylene, propylene, styrene, vinyl chloride, vinyl acetate, vinyl alcohol, vinylidene chloride, carbonate, amide, ethylene terephthalate, and ethylene-vinyl acetate. The term “packaging polymer” refers to a composition comprising two or more packaging monomers. A packaging monomer or packaging polymer can be used to form the packaging material. In an embodiment, the packaging material can be used to form the container, structure, or the like.

### Discussion:

Embodiments of the present disclosure provide for biosensors that include a material, such as polydiacetylene (PDA) material, where the material is used for detection of a microbe or microbial product present in a fluid present in the container. Embodiments of the present disclosure provide for biosensors that include a material such as PDA material, where the material is used for detection of a microbe or microbial product present in a fluid present in the container. In an embodiment, the present disclosure provides for containers, or structures used in conjunction with the containers, that include a PDA material, where the PDA material is used for detection of a microbe or microbial product present in a fluid present in the container. In an embodiment, a change of PDA color (e.g., blue to red) indicates detection of the microbe or microbial product in the fluid within the container. In an embodiment, the PDA material can be selected and/or the container or structure designed so that only certain types of microbes can be detected or so that a plurality of types of microbes is detected.

Although many of the embodiments refer to PDA, other chemicals can be used that undergo a color change upon interaction with a microbe or microbe products. Also, many embodiments refer to a container, but those descriptions can refer to biosensors, and are not limited to containers. A portion of the discussion describes containers and PDA materials, but embodiments of the present disclosure are not limited to either containers or PDA materials.

PDA materials can be used since a color change occurs when the PDA monomers crosslink. In particular, the PDA monomers appear as an intense blue color owing to their conjugated ene-yne framework, and upon interaction with the microbe or microbial product, a conformational transition occurs in the conjugated polymer backbone leading to intense blue-red color changes. This color change can be used to as an indicator of the presence of the microbe or microbial product. In particular, the color change is caused by external structural perturbations, such as binding of amphiphilic and bacterial membrane associated hydrophobic molecules causes conformational transitions in the conjugated polymer backbone.

In an embodiment, the container or structure has a PDA material incorporated therein or disposed on a surface of the container or structure. The container and structure can be made of the same material or of different materials. In an embodiment, a packaging or other monomer and a PDA material (e.g., diacetylene monomer) can be mixed and polymerized prior to formation of the container or structure. In an embodiment, formation of the container or structure includes curing and molding the material into a desired shape. A desired shape for a container can include a container bottle or other type of container as well as caps or nozzles that can be disposed on the container body, while shapes of the structure are described in more detail below.

The following describes methods of preparing various embodiments of the present disclosure. In preparing the materials having a PDA material incorporated therein, diacetylene monomers and packaging or other monomers can be mixed in organic solvent/s, aqueous solutions, or mixtures. In an embodiment, the following variables can be modulated: solvent type, ratio between the monomers, and addition of additives required for plastic properties. The diacetylene monomers and packaging or other monomers can then be polymerized. In an embodiment, the following variables can be modulated: separate polymerization of components/simultaneous polymerization; degree of polymerization; and polymerization before/after molding. In an embodiment, the polymerization of the PDA material can be controlled using UV light at about 254 nm. In an embodiment, the PDA material and packaging or other polymers can then be molded to the desired shape/structure and curing/annealing. In an embodiment, the following variables can be modulated: duration of curing; temperature; and post-curing polymerization steps (See Example 7).

In an embodiment, the material used to form the container or structure can also include hydrolyzed silica or metallic nano/microparticles such as gold, silver, copper or inorganic nano/microparticles such as zinc oxide, titanium oxide (See Example 7). In an embodiment, a packaging or other monomer, a diacetylene monomer, and a silica precursor or micro/nanoparticle, are mixed prior to formation of the packaging material. In an embodiment, the silica precursors can include, but are not limited to, tetraethyl orthosilicate (TEOS), tetramethyl orthosilicate (TMOS), methyltrimethoxysilane (MTMS), diethoxydimethylsilane (DEMS), vinylotriethoxysilane (VTES), and combinations thereof. In an embodiment, the silica can be included to produce a rough surface, which can enhance adhesion of the microbe to the surface.

In an embodiment, diacetylene monomers are mixed with silica precursors. In an embodiment, the following variables can be modulated: ratios among components; type of silica precursors; and nature of solvents. Packaging monomers are then dissolved in appropriate solvents. Parameters to be modulated are polymer preparation protocols. The two monomer solutions can then be mixed. In an embodiment, the following variables can be modulated: timing of reagent addition and mixing; temperature; and ratios. The mixture can then be molded to desired shapes and structures, and cured and polymerized. In an embodiment, the following variables can be modulated: the order of the two processes; and duration. In some embodiments, a mixed assembly is created through thin film techniques (e.g., dip-coating, layer-by-layer, nano/micro imprinting, ink-jet printing, lithography or spin coating (See Example 7)).

In an embodiment, a packaging or other polymeric material is made using a process comprising the steps of: 1) mixing a diacetylene monomer with a silica precursor (first solution), 2) mixing the first solution with a packaging or other monomer to form a second solution, and 3) polymerizing the second solution. In another embodiment, a packaging or other polymeric material is made using a process comprising the steps of: 1) mixing a diacetylene monomer, a silica precursor and a packaging or other monomer, and 2) polymerizing the mixture.

In an embodiment where the PDA material (e.g., PDA monomer or unpolymerized PDA) is included into the manufacturing process of the container, the container can be sterilized without affecting the PDA material. After sterilization, the PDA monomer can be flash polymerized using a high intensity laser to activate the PDA material.

In an embodiment, the polymerization time of the PDA monomer can be controlled to optimize the colorimetric response of the PDA material. Optimization of the PDA monomer can be conducted using UV-Vis spectrophotometry. The absorbance values at 530 and 650 nm can be recorded on an interval (e.g., about 5 seconds). The earliest time point at which the ratio reaches a stable value was determined as the optimized polymerization time.

Having described some embodiments for making the container and structure, focus is now directed towards to details regarding the PDA material. In an embodiment, the PDA material can be disposed on the entire container or structure, or any portion of a container or structure. In one embodiment, the PDA material can be disposed on one side of container or structure that contacts the fluid in the container, a neck or lip portion of the container, and the like. In one embodiment, a PDA material is coated onto the neck or lip portion of the container.

In an embodiment, the PDA material can be disposed via multiple appropriate techniques including, but not limited to, dip coating, aerosol coating, coating with monolayers prepared at the air/water interface, nano/micro imprinting technology, ink-jet printing, lithography technology methods, and the like (See Example 7). In an embodiment, disposing can include the application of a single layer of PDA material, multiple layers of PDA materials (identical or different types of PDA materials), or multiple layers of PDA material and other materials.

In an embodiment, a container or structure is coated with a PDA material includes 10,12-tricosadiynoic acid, tetraethyl orthosilicate, nitric acid, and water. The mole ratios of the 10,12-tricosadiynoic acid, tetraethyl orthosilicate, nitric acid, and water can be approximately 1:9:312:0.13:0.05, respectively.

In an embodiment, the structure may be attached to the container or can be added to or within the container. In an embodiment, the structure can be a polygonal object, a flat disk, a filter, a spherical object, a spherical porous sphere containing PDA vesicles or micelles, or multiple PDA spheres sensitive to different pathogens where the PDA material is disposed on the surface of the structure so that fluid of the container can be exposed to the microbe or microbial products. In an embodiment, the structure has a roughened surface or the structure does not have a smooth surface, where the non-smooth surface may increase adherence of the microbe or microbial product to the structure. In an embodiment, the structure can vary in size from the mm range to cm range. In regard to multiple PDA spheres sensitive to different pathogens, it is advantageous that certain types of PDA materials are more or less sensitive to certain microbes.

In an embodiment, the structure can be made of a material such as glass, a nitrocellulose membrane, poly(methyl methacrylate) (PMMA) substrate, a cellulose acetate substrate, and polyurethane where the PDA material is disposed on the surface of the structure.

In an embodiment, the structure can be porous so that the PDA material and fluid can interact with one another. For example, the porous structure can be impregnated with the PDA material and/or the PDA material can be disposed within the pores of the porous material. In an embodiment, the porous structure can be made of a material such as agar, cellulose acetate, a solgol, polyurethane and a combination thereof. In embodiment, the structure can be a porous, opaque substrate so that the color change may be more readily observable. In an embodiment, the porous, opaque substrate can be made of a nitrocellulose membrane.

In a particular embodiment, a PDA-based ball-like structure can be inserted into the container with the fluid. In an embodiment, the PDA-based ball-like structure can respond to the existence of the microbe or microbial product by changing color. In an embodiment, the PDA-based ball-like structure includes a PDA material and is physically large enough so as to not squeeze through the opening of the container where the fluid is dispersed. In an embodiment, the PDA-based ball-like structure can include a filter-type interface having a pore size small enough to capture microbes (e.g., which may bring the microbe in close proximity with the PDA material, thereby amplifying the effective concentration of the microbe in the vicinity of the PDA material, leading to color change). In an embodiment, the PDA-based ball-like structure can also be created from a perforated material that can either increase the effective surface area of the ball-like structure and/or allow for trapping microbes that diffuse to the area of the ball-like structure through simple diffusion. In an embodiment, the PDA-based ball-like structure could be made of any of the material described herein, and in particular, can be made of a plastic or polymeric material (perforated or not) and the PDA material can be attached to its surface with proper surface functionalization.

In an embodiment, the PDA-based ball-like structure could report the detection of microbe or microbial products through a color change, through a fluorescence signal, through an electric signal, and/or a radio frequency identification (RFID) tag that is implanted in it. If a color change is sought after, the contrast can be made twice as high if one hemisphere of the PDA-based ball-like structure is covered by an optically reflective surface. In this way a ray of light would travel through the PDA-based ball-like structure twice before reaching the eye of an observer, thereby increasing the contrast by two fold. In another embodiment, the same could be made for an arbitrary coverage of the ball by reflective covers, either continuous or randomly located throughout the PDA-based ball-like structure surface.

In an embodiment, the PDA material can be encapsulated in a gel/hydrogel form (e.g., agarose) and then encapsulated within a thin membrane or film that covers it to render a ball-like structure, or covered by a filter-type interface. In another embodiment, the PDA material could be bound (e.g., covalent bond, ionic bond, electrostatic bond, and the like) to a plastic surface, thereby reducing the chance of PDA material leaking to the container.

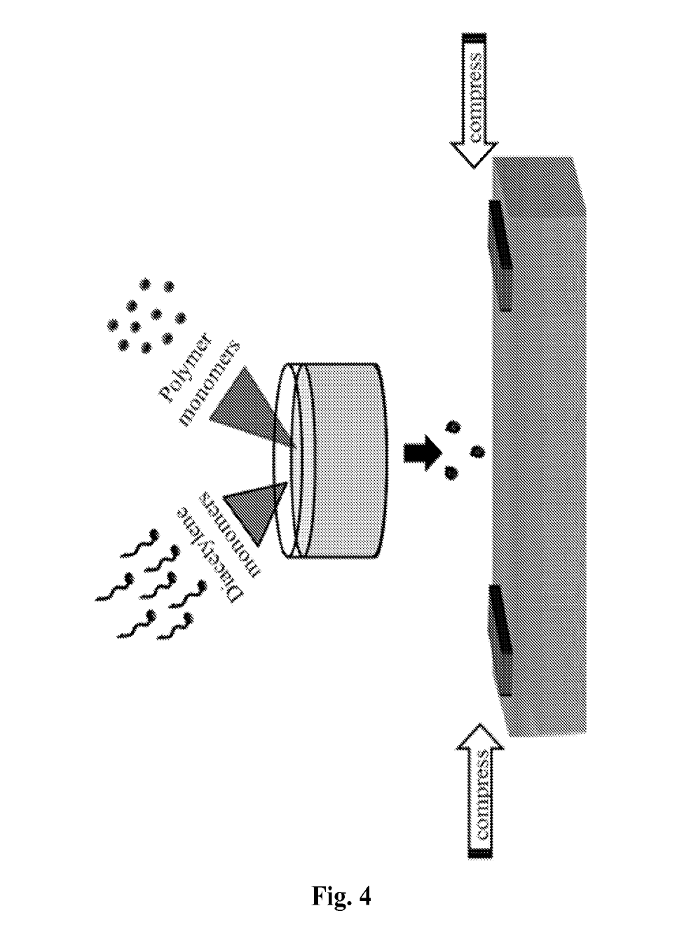

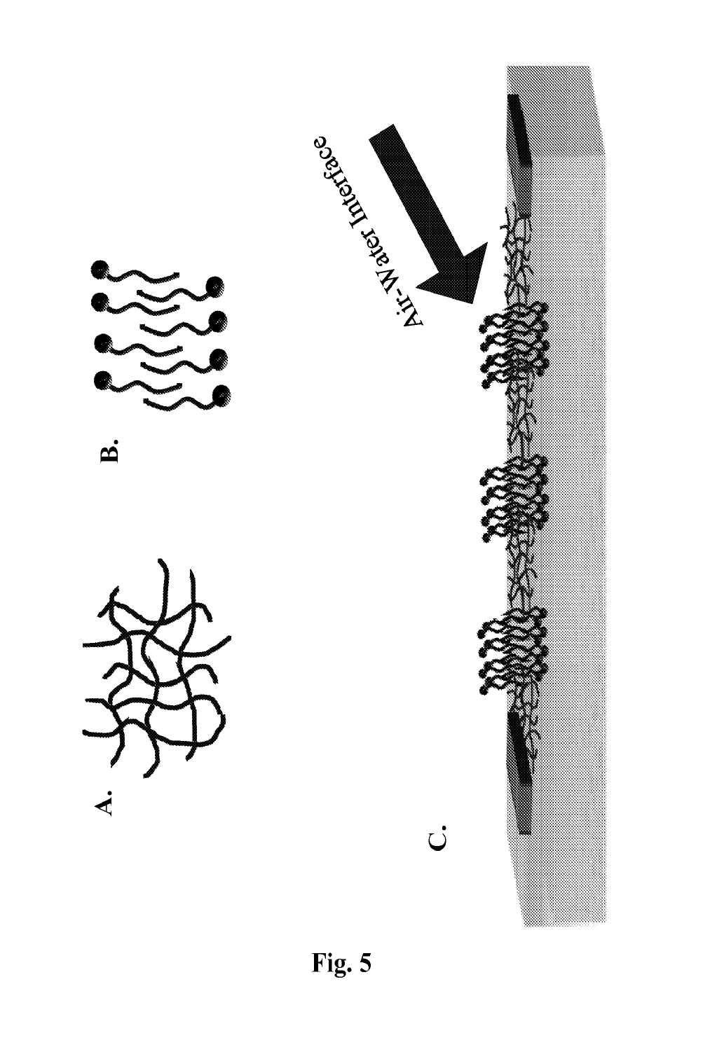

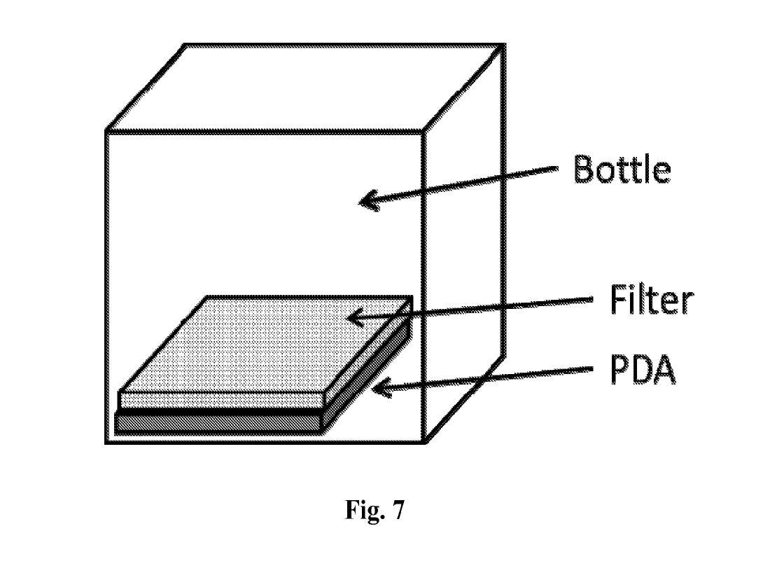

In an embodiment, a filter having a certain size pore specific for one or more types of microbes could be used to filter microbes from the fluid in the container (See, FIG. 7). In an embodiment, a filter of approximately 0.2 μm could be used to filter microbes (e.g., bacterial cells are 0.2-5 μm in size) from the fluid in the container. In this regard, a 0.2 μm filter (which resembles a mesh) (or a similar filter having a different pore size for other microbes) can be coated with a thin layer of PDA material, a PDA-absorbed gel, or individual PDA materials, embedded PDA vesicles or micelles such that the filter is still acting as a filter and thereby capable of capturing microbes such as bacteria on its surface. Upon capturing of bacteria by the filter, the PDA material is effectively seeing a much higher concentration of bacteria in its vicinity than otherwise represented by the concentration of microbe in the fluid, which can amplify the PDA material signal. In an embodiment, the filter can be positioned along the internal walls, at the bottom of a container, or at the top of the container near the opening. In another embodiment, the PDA material can be disposed within a gel and the filter is positioned adjacent to or around the PDA-gel (See FIG. 4). In an embodiment, sol-gel/PDA films can also be prepared at the air/water interface, i.e. using the Langmuir method and/or a method generally shown in the schematics of FIGS. 4 and 5. These sol-gel/PDA films are then transferred onto the packaging or other substrate. Polymerization can be carried out prior to film transfer or after.

In another embodiment, the PDA material is attached to the output of a microfluidic device within the container that filters and segregates bacteria to the PDA material. Segregation can be size-based—leading to a concentration of bacterial cells at the filter matrix and a subsequent induction of color change in the filter-associated PDA material. In an embodiment, the microfluidic device could employ a combination of channels within the container at decreasing widths to accommodate bacteria at all sizes at the beginning and as the liquid flows through, bacteria would get trapped in the channels depending on their size. In an embodiment, the PDA material can be coated at one or more positions along the channel so that the bacteria can interact with the PDA material.

In an embodiment, the PDA material is naturally fluorescent in visible colors upon activation by the microbe or microbial products. In an embodiment, the PDA material can be visualized using a light source (as part of the container, e.g., in the container cap) that would excite the fluorescent PDA as needed. In this embodiment, a light source of low weight and dimension (e.g., LED, laser diode etc.) is powered by a small power source (e.g., any battery, flat battery, or paper-based battery), which may be trigged upon manual or automatic switch (e.g., squeezing the cap). The light is directed towards the PDA-containing object, and the fluorescent light from the PDA, if any, is seen by the observer.

In an embodiment, the container can include one or more sub-compartments that are separate from the main compartment of the container. In an embodiment, the sub-compartments can be located on the sides of the container or bottom of the container. The PDA material or structure including the PDA material can be included in the sub-compartment. In an embodiment, the fluid in the main compartment of the container can come into contact with the PDA material upon an event such as removal of a seal, opening of the cap of the container, or removal of a tab, so that the PDA material comes into contact once the seal is broken or the cap is turned past a certain point. In an embodiment, the event can cause a portion of the sub-compartment to open to the fluid in the main compartment. In an embodiment, the container may need to be shaken or otherwise mixed to ensure that the PDA material and the fluid come into contact with one another.

In an embodiment, the container includes a tab that when removed exposes the fluid in the container to the PDA material (e.g., the PDA can be in a sub-compartment or the tab should separate the PDA material from the fluid). In an embodiment, the tab can be disposed on the side of the container, in the cap of the container, and the like. Once the tab is removed, the fluid (and microbes or microbial products therein) can contact the PDA material exposed by removal of the tab. In an embodiment, the PDA material and/or the substrate including the PDA material can be any of those described herein.

In an embodiment, the container includes two sub-compartments (a secondary compartment and a tertiary compartment). In an embodiment, the secondary compartment includes the PDA material, while the tertiary compartment includes a PDA activation solution. In an embodiment, the PDA activation solution is an acid, base, surfactant, organic components, micro/nano particles, gaseous components (e.g., carbon dioxide or nitrous oxide) or other material that causes the PDA material to undergo a color change. In an embodiment, there is a boundary between the secondary compartment and the tertiary compartment, where the boundary can be dissolved by the microbe or microbial product. If a microbe or microbial product is present in the fluid in the main compartment of the container, the microbe or microbial product dissolves the boundary and causes the PDA material and the activating material to come into contact with one another causing indirect, rapid color change in the PDA.

Now having described the container, structure, and embodiments of ways to incorporate the PDA material into the container or structure, attention is now directed toward the specific PDA materials. In an embodiment, the PDA material can include a PDA layer or film, a PDA nanoparticle, a PDA vesicle, a PDA micelle, or a combination thereof. In particular the PDA material can be a PDA nanoparticle.

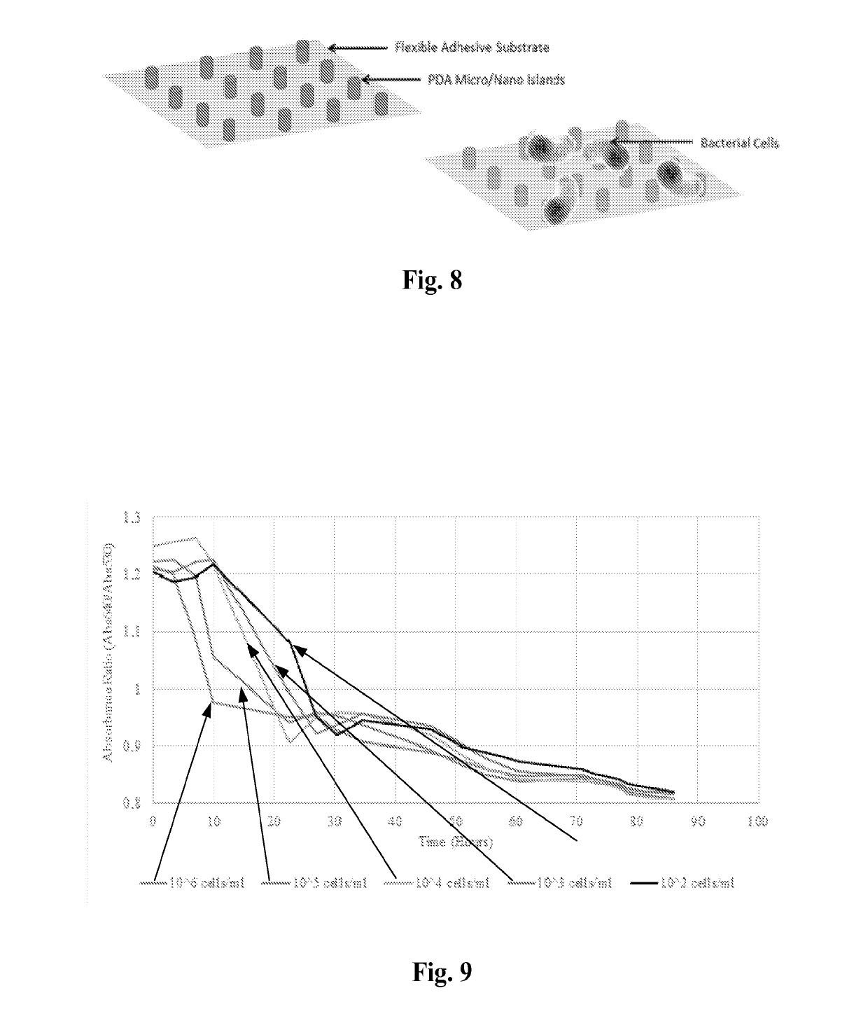

In an embodiment, the PDA layer or PDA film can be disposed directly onto the surface of the container or a structure within the container. In an embodiment, shown in FIG. 8, the PDA layer or PDA film can be continuous or discontinuous (e.g., including one or more islands etc.) on the surface of the container or structure. In an embodiment, PDA micro/nano islands can be printed on the substrate using ink-jet printing or nano/micro imprinting technology. In an embodiment, the substrate can be chosen or modified to contain certain surface or charge properties conducive to printing. In an embodiment, the size of the islands could vary from about 100 nm to 1 cm, about 100 to 500 nm, about 500 nm to 100 μm, about 500 nm to 1 μm, diameter or in width, length and/or height. In an embodiment, the shape of the micro/nano islands could be circular, pyramidal, polygonal, and the like such as to provide surface roughness and optimal bacterial binding capabilities. In an embodiment, the islands can be nanoparticles, as described herein. In an embodiment, the PDA layer or PDA film can have a thickness of about 1 μm to 1 cm and a width and length appropriate to cover the desired area of the container (e.g., 100 μm to 10 cm).

In an embodiment, the PDA nanoparticle, the PDA vesicle, and/or the PDA micelle can be attached (e.g., covalently, ionically, electrostatically, etc.) to the surface of the container and/or structure, randomly or in an ordered fashion (e.g., an array) using alternately charged polymers such as polyacrylic acid, polystyrene sulfonate, linker molecules, or a salinization inducing agent such organofunctional alkoxysilane molecules. In an embodiment, the PDA nanoparticle, the PDA vesicle, and/or the PDA micelle, can form a layer of PDA nanoparticles, of PDA vesicles, and/or of PDA micelles, where the layer is distinct form a film layer. In an embodiment, the PDA nanoparticle can be formed on the container or structure using a micro/nano imprinting technology, or ink-jet printing. In an embodiment, the PDA nanoparticle, the PDA vesicle, and/or the PDA micelle, can be disposed within a porous structure or a filter structure so that fluid can still contact the PDA material.

In an embodiment, a PDA nanoparticle can include a particle having a longest dimension of about 1000 nm or less, about 500 nm or less, about 250 nm or less, about 100 nm or less, or about 50 nm or less and/or a shortest dimension of about 100 nm or less, about 50 nm or less, about 25 nm or less, about 10 nm or less, or about 5 nm or less, and all ranges between the longest and shortest dimensions. The PDA nanoparticle can be a PDA nanosphere, a non-spherical PDA nanoparticle, a PDA nanowire, a PDA nanotube, a PDA nanosheet, a PDA nanoribbon, and the like. The PDA nanowire, PDA nanotube, or PDA nanoribbon, can have a diameter of about 1 to 100 nm and a length of about 10 to 500 nm. The PDA nanosheet can have a length and/or width of about 10 to 500 nm and a thickness of about 1 nm to 20 nm. The PDA nanosphere can have a diameter of about 5 to 500 nm.

In an embodiment, the PDA nanoparticle can include a particle having a longest dimension of about 1000 nm or less, about 500 nm or less, about 250 nm or less, about 100 nm or less, or about 50 nm or less and/or a shortest dimension of about 100 nm or less, about 50 nm or less, about 25 nm or less, about 10 nm or less, or about 5 nm or less and all ranges between the longest and shortest dimensions. In an embodiment, the PDA nanoparticle can be a PDA nanosphere, a non-spherical PDA nanoparticle, a PDA nanowire, a PDA nanotube, a PDA nanosheet, a PDA nanoribbon, and the like. The PDA nanowire, PDA nanotube, or PDA nanoribbon, can have a diameter of about 1 to 100 nm and a length of about 10 to 500 nm. The PDA nanosheet can have a length and/or width of about 10 to 500 nm and a thickness of about 1 nm to 20 nm. The PDA nanosphere can have a diameter of about 5 to 500 nm. The non-spherical PDA nanoparticle can have a longest dimension of about 5 to 500 nm.

In some embodiments, the PDA material is contained within a vesicle or micelle and incorporated into a material used to form the container or structure or disposed on a portion of the container or structure. In an embodiment, the vesicle can include a lipid, glycoprotein, antibody, aptamer, or sugar, PDA vesicle such as those described in U.S. Pat. No. 7,794,968 and U.S. Pat. No. 8,008,039. In an embodiment, a packaging or other polymeric material used to from the container or structure can be formed by a process comprising the steps of: 1) dissolution of a diacetylene monomer in an aqueous solution to result in formation of a PDA vesicle or micelle (first solution), 2) dissolution of a packaging or other monomer in a mild organic solvent (second solution), 3) mixing the first and second solutions to form a third solution, 4) using ultrasonication to form vesicles or micelles, and 5) polymerizing the third solution.

In an embodiment, the PDA vesicle refers to a spheroidal, elliptical or cylindrical micro-particle platform comprising of double-chain phospholipids and polymerized PDA. PDA-vesicle wall can include of bilayer leading to a hydrophilic core and exterior. In an embodiment, the PDA vesicle can have a diameter of about 100 nm to 1000 μm.

In an embodiment, the PDA micelle refers to a PDA vesicle that includes a spheroidal, elliptical or cylindrical micro-particle platform including of single-chain phospholipids and polymerized PDA. In an embodiment, the PDA-micelle wall can include a monolayer leading to a hydrophobic core and hydrophilic exterior. In an embodiment, the PDA micelle can have a diameter of about 10 nm to 500 μm.

In an embodiment, an agent can be bound to the PDA material and/or can be disposed adjacent the PDA material to enhance the interaction of the microbe or microbial products with the PDA material. In an embodiment, the agent can include a capturing agent, a charged material, or a combination thereof.

In an embodiment, the capturing agent can be attached to the PDA material. The capturing agent binds to the microbe. In an embodiment, the capturing agent can include: a sugar, a glycoprotein, an antibody, an aptamer, metallic nanoparticle, and a combination of mentioned agents. In an embodiment, the capturing agent is bound to the PDA material through a covalent, ionic, or electrostatic bond. In an embodiment, the capturing agent is bound to a surface of the container or substrate so that the capturing agent is adjacent (e.g., in close proximity) the PDA material, so that the microbe or microbial product can interact with the PDA material.

In an embodiment, a charged material can be bound to the PDA material or can be disposed adjacent the PDA material to enhance the interaction of the microbe or microbial products with the PDA material. In an embodiment, the charged material (e.g., ions, polymers, nanoparticles) can be attached to the PDA material. The charged material attracts an oppositely charged microbe. In an embodiment, the charged material can include: polymers such as polyacrylic acid, polystyrene sulfonate, or metallic/inorganic nanoparticles and monovalent or divalent salts. In an embodiment, the charged material is bound to the PDA material through a covalent, ionic, or electrostatic bond. In an embodiment, the charged material is bound to a surface of the container or substrate so that the charged material is adjacent (e.g., in close proximity) the PDA material so that the microbe or microbial product can interact with the PDA material.

In an embodiment, the PDA material can be used in conjunction with a fluorescent material, a dye, and/or a quenching material, to enhance the change that the PDA material undergoes upon exposure to the microbe or microbial product.

The PDA material that is not exposed to bacteria (herein referred to as inactivated PDA material) is blue (i.e., has high optical absorption anywhere but in the blue optical regime). The PDA that is exposed to bacteria (herein referred to as activated PDA material) is red (i.e., has high optical absorption anywhere but in the red optical regime). In an embodiment, it may be difficult to visualize dim PDA material color changes in a semi-transparent plastic as the environment background is colorful. The visual clues of the color change can be enhanced by creating contrast that is not only based on color but also based on overall intensity of light getting to the eye of the observer, i.e., red light versus no light (i.e., black), or blue light versus no light.

In one embodiment, an optical dye can be used with (e.g., mixed with or attached to or near the PDA material) the PDA material that absorbs in the red regime (e.g., QSY21 by Invitrogen). While inactivated PDA material will look the same (blue), activated PDA material will appear dark (as the PDA will absorb anywhere but the red, and the optical dye will absorb the red). In another embodiment, an optical dye can be used with (e.g., mixed with or attached to or near the PDA material) the PDA material that absorbs in the blue regime (e.g., QSY35 by Invitrogen). Inactivated PDA material will look dark (as the PDA material will absorb anywhere but the blue, and the optical dye will absorb the blue), activated PDA material will appear normal red. For example a dye is introduced that absorbs all visible spectrum apart from deactivated PDA material such that without microbial detection a container cap head is black; with microbial detection, the container cap head is colored. A dye can also be introduced that absorbs all visible spectrum apart from activated PDA material such that without microbial detection a container cap head is colored; with microbial detection a container cap head is black.

In another embodiment, when using a fluorescent PDA material, the deactivated (or activated) PDA material signal is preferentially quenched (or enhanced) by attaching it close to the surface of a quenching material (for example, gold nanospheres (about 532 nm), gold nanorods (550-700 nm) with peak absorbance overlapping with the deactivated (or activated) form of PDA material.

In another embodiment, a fluorescent dye is added to the PDA material. In still another embodiment, an enzymatic substrate is coupled with the PDA material such that the PDA color change is coupled to an enzymatic reaction that also produces color such as an HRP reaction.

An embodiment of the present disclosure also includes detecting one or more microbe or microbial products in a container. More particularly, included herein is a method for detecting one or more microbe or microbial products in a container, which includes contacting the fluid with the PDA material, where a color change in the PDA material indicates detection of a certain level of microbe or microbial products and/or a type(s) of microbe or microbial product. In an embodiment, the fluid in the container comes into contact with a portion of the container (e.g., a wall, an interior, a cap, a compartment) or a substrate (e.g., disposed within the container) associated with the container. As noted herein, the PDA material can be directly within the container material or substrate or disposed on the surface of the container or substrate.

It should be understood that the foregoing relates to preferred embodiments of the present disclosure and that numerous changes and combinations of various embodiments may be made therein without departing from the scope of the disclosure. The disclosure is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present disclosure and/or the scope of the appended claims.

### EXAMPLES

### Example 1

### Preparation of Glass PDA and Perspex PDA to Study Bacteria Colorimetric Response

This Example describes the results obtained after execution of “PDA Testing Protocol Psuedomonas+Staphyloccocus”. Briefly, the test was carried out to measure the colorimetric response induced in polydiacetylene (PDA) coated circular Perspex and square glass substrates under the influence of Pseudomonas Aeruginosa and Staphylococcus Aureus. Two distinct chemical formulations of PDA were tested using Pseudomonas Aeruginosa and Staphylococcus Aureus incubated with growth medium and phosphate buffered saline (PBS). Five distinct concentrations (10̂6, 10̂5, 10̂4, 10̂3, 10̂2) of each bacteria type were tested and colorimetric response was measured at regular intervals. Details related to test setup, testing methods and data analysis have been described in “PDA Testing Protocol Psuedomonas+Staphyloccocus”.

### Acceptance Criteria

Results were interpreted as follows:
1) The colorimetric response (CR) was measured for PDA sensors based on the absorbance at 640 nm and 530 nm. In order to correctly estimate the CR, the absorbance values should be large than 0.1 after subtracting the background.
2) Color change was measured for PDA sensors based on the ratio of absorbance measured at 640 nm and 530 nm (Abs640/Abs530).
3) Visual color change observed (blue to red) in the PDA sensors based on subjective observation.

### Testing Summary

### Results

After 87 hours of real-time colorimetric response measurement, the following conclusions can be drawn based on visual observation of test plates:
PDA sensors (both glass PDA and Perspex PDA) did not change color under the influence of Staphylococcus Aureus bacteria (incubated in growth media and PBS) in 87 hours since experiment started The data acquired was not processed to obtain CR and Abs640/Abs530 values as no color change was observed based on subjective assessment.
PDA sensors (both glass PDA and Perspex PDA) changed color under the influence of Pseudomonas Aeruginosa incubated in growth medium.
PDA sensors (both glass PDA and Perspex PDA) did not change color under the influence of Pseudomonas Aeruginosa incubated in PBS. CR and Abs640/Abs530 values were not calculated as no color change was observed based on subjective assessment.

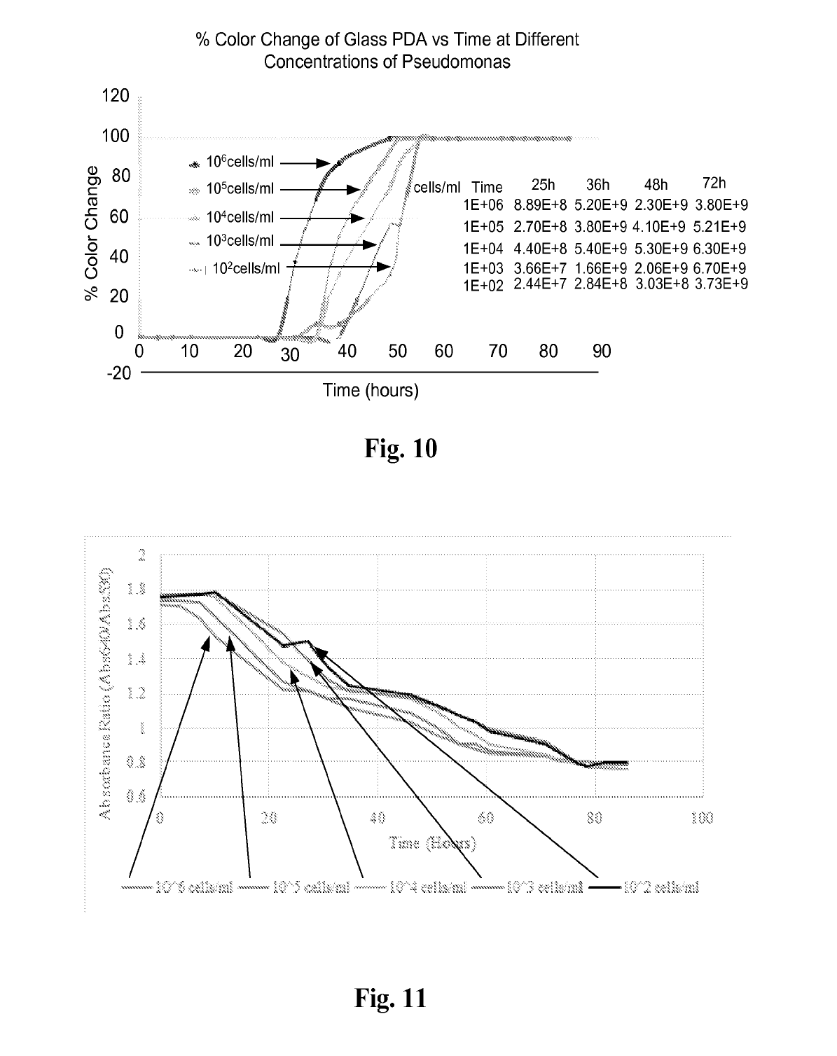

Color change values (ratios of Abs₆₄₀/Abs₅₃₀) was plotted against time as shown in FIG. 9 and % color change of glass PDA was plotted against time as shown in FIG. 10, each for Plate 3.

FIG. 10 illustrates the % color change of glass PDA vs. time at different concentration of Pseudomonas Aeruginosa in growth medium. (Inset: Concentration of Pseudomonas Aeruginosa in growth medium at different time points).

Table 1 summarizes sensor response observations based on FIG. 10.

TABLE 1
Details related to the colorimetric response of glass-PDA sensors due
to Pseudomonas Aeruginosa incubated in growth media.

| Initial | | | | |
|---|---|---|---|---|
| Pseudomonas | Time at which | Bacterial | Time at which | Bacterial |
| Aeruginosa | initial response | Concentration at | final response | Concentration at |
| Concentration | was observed | initial response | was observed | final response |
| (cells/ml) | (hours) | (cells/ml) | (hours) | (cells/ml) |
| 10\hat{ }6 | 27 | 10\hat{ }8 to 10\hat{ }9 | 50 | 10\hat{ }9 |
| 10\hat{ }5 | 36 | 10\hat{ }9 | 55 | 10\hat{ }9 |
| 10\hat{ }4 | 36 | 10\hat{ }9 | 55 | 10\hat{ }9 |
| 10\hat{ }3 | 50 | 10\hat{ }9 | 58 | 10\hat{ }9 |
| 10\hat{ }2 | 50 | 10\hat{ }8 | 58 | 10\hat{ }9 |

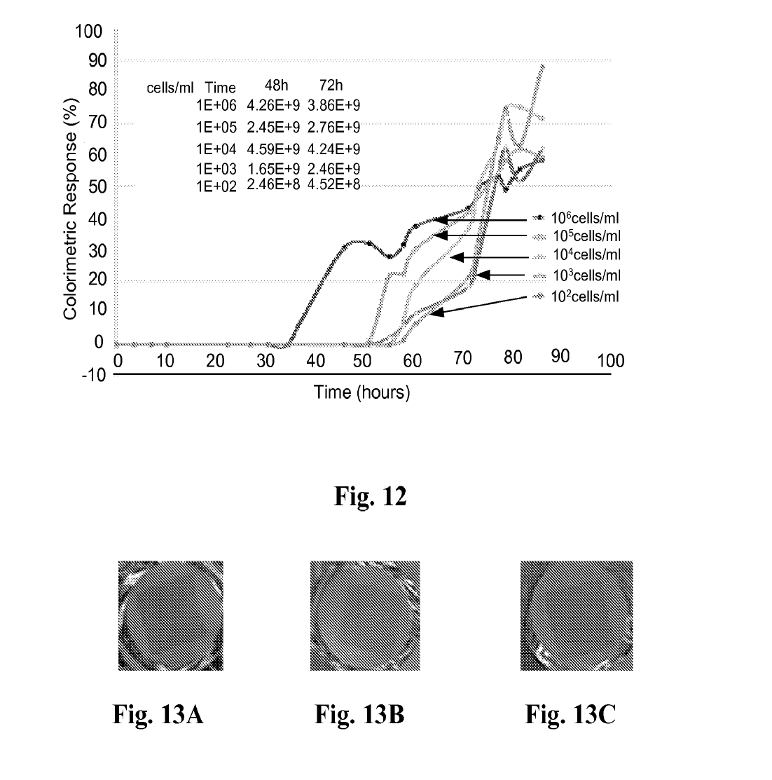

Color change values (ratios of Abs₆₄₀/Abs₅₃₀) was plotted against time as shown in FIG. 11 and CR was plotted against time as shown in FIG. 12 for Plate 4.

FIG. 11 illustrates the color change values (ratios of Abs₆₄₀/Abs₅₃₀) measured in Perspex PDA sensors vs. time at different concentrations of Pseudomonas Aeruginosa. FIG. 12 illustrates the CR measured in Perspex PDA sensors vs. time at different concentrations of Pseudomonas Aeruginosa. (Inset: Concentration of Pseudomonas Aeruginosa in growth medium at different time points).

Table 2 summarizes sensor response observations based on FIG. 12.

TABLE 2
Details related to the colorimetric response of Perspex PDA sensors due to
Pseudomonas Aeruginosa incubated in growth media.

| Initial | | | | |
|---|---|---|---|---|
| Pseudomonas | Time at which | Bacterial | Time at which | Bacterial |
| Aeruginosa | initial response | Concentration at | final response | Concentration at |
| Concentration | was observed | initial response | was observed | final response |
| (cells/ml) | (hours) | (cells/ml) | (hours) | (cells/ml) |
| 10\hat{ }6 | 35 | 10\hat{ }8 to 10\hat{ }9 | 55 | 10\hat{ }9 |
| 10\hat{ }5 | 38 | 10\hat{ }8 to 10\hat{ }9 | 62 | 10\hat{ }8 to 10\hat{ }9 |
| 10\hat{ }4 | 55 | 10\hat{ }9 | 62 | 10\hat{ }9 |
| 10\hat{ }3 | 56 | 10\hat{ }9 | 72 | 10\hat{ }9 |
| 10\hat{ }2 | 56 | 10\hat{ }8 | 73 | 10\hat{ }9 |

Bacterial counts and absorbance values were recorded for Plate 3 at the following time points 3 h, 10 h, 25 h, 36 h and 49 h.

Bacterial counts and absorbance values were recorded for Plate 4 at the following time points 3 h, 10 h, 23 h, 49 h, 55 h, 62 h and 73 h.

### Discussion

Results obtained for Plate 3 (PDA coated square glass incubated with Pseudomonas Aeruginosa in growth media.

The absorbance values obtained for Plate 3 at 530 nm and 640 nm after background subtraction were less than 0.1. High background noise was obtained due to Pseudomonas Aeruginosa overgrowth (greater than 10̂7 cells/ml) resulting in incubation solution turbidity. Pseudomonas Aeruginosa are known to form biofilms which could also result in higher turbidity. A clear growth medium was observed at 6.5 h and turbid growth medium at 34.5 h, respectively. Moreover, PDA coated glass sensors displayed low absorbance intensities at 530 nm and 640 nm, due to the sensor design and fabrication decisions. The color intensity remained low and did not induce high absorbance even after the bacteria induced a blue to red colorimetric response. Thus, the compounding effect of the high background absorption and low signal absorbance (intensity of PDA coated glass sensors) resulted in non-significant CR values. FIG. 9 represents color change values (ratios of Abs₆₄₀/Abs₅₃₀) measured for glass PDA sensors obtained by evaluating spectrophotometry data related to Plate 3. The figure shows that the absorbance at blue (Abs640) is decreasing while the absorbance at red (Abs530) is increasing as time progress, indicating the glass PDA sensors are changing color from blue to red.

Although the CR measurement could not be performed for plate 3 the method remains valid and should work for the targeted bacteria concentrations (10̂2˜10̂⁶ cells/ml), since the growth medium remains clear at these concentrations.

In order to obtain colorimetric response for Plate 3, the color change was subjectively judged based on the digital photographs of Plate 3 taken at different time points.

Based on the images the following assessment scheme was developed to manually judge the color change.

If the PDA sensor did not change color (remained completely blue), 0 points were assigned to the particular sensor. A representative example is shown in FIG. 13A.

If the PDA sensor displayed an intermediate blue-red (purple) color or if half the sensor area had changed color completely to red, 0.5 points were assigned to the particular sensor. A representative example is in FIG. 13B.

If the PDA sensor completely changed color (became red), 1 point was assigned to the particular sensor. A representative example is shown in FIG. 13C.

The digital photographs of glass-PDA sensors obtained before (0 hours) and after (127 hours) treatment with bacteria were used as reference standard for blue and red color.

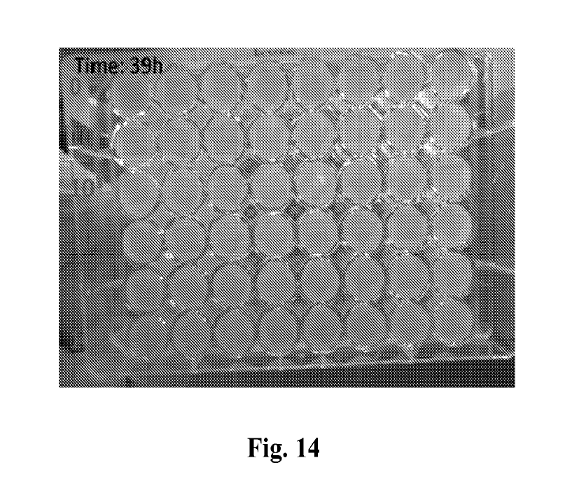

For example: Digital photograph of Plate 3 captured at 39 h is FIG. 14. Based on a careful examination of the plate it can be observed that the sensors present in the 10̂6 cells/ml row are completely red and would be scored as 1. Sensors in rows with 10̂4 and 10̂5 cells/ml concentration would be scored as 0.5. The sensors in the 0 and 10̂2 cells/ml rows would be scored at 0.

This subjective estimation of color was conducted by staff scientist experienced in working with PDA sensors. The confidence level in assigning 0 and 1 points are higher than assigning 0.5 points, since it is easier to determine if the PDA sensor is completely blue or red. If all the wells in one bacteria concentration treatment group change color to red, then 8 points will be assigned to this group.

The formula to calculate color change based on percentage of well area is shown below:

$$\% color change in P D A - Glass = \frac{Total Points Assigned}{8} \times 100$$

Total Points Assigned—Sum of Points assigned for Individual Bacteria Concentration Group at Specific Time Point

For example: If 10̂6 cells/ml bacteria concentration has 4 wells and half of one well (based on well area) change color to red (determined subjectively) at 30 h, then 4.5 points will be given to this bacteria concentration group at 3 hours, and the % Color Change=4.5/8=56.25%

FIG. 10 shows the scatter plot obtained by graphing % Color Change against time. From results summarized in Table 1, we can estimate that PDA-glass sensors displayed a concentration dependent colorimetric response in the presence of Pseudomonas Aeruginosa. A threshold bacterial concentration of 10̂8˜10̂9 cells/ml is required to induce colorimetric response.

Results obtained for Plate 4 (PDA coated Perspex incubated with Pseudomonas Aeruginosa in growth media.

Issues related to bacterial overgrowth were also observed in Plate 4 (clear growth medium and turbid growth medium). However, Perspex PDA sensors display high absorbance values at 530 nm and 640 nm. Thus, the signals obtained after background subtraction were greater than 0.1 and enabled accurate calculation of CR and color change. FIG. 11 represents color change values (ratios of Abs₆₄₀/Abs₅₃₀) measured in Perspex PDA sensors obtained by evaluating spectrophotometry data related to Plate 4. FIG. 11 shows that the absorbance at blue (Abs640) is decreasing while the absorbance at red (Abs530) is increasing as time progress, indicating the Perspex PDA sensors are changing color from blue to red. From results summarized in Table 2, we can conclude that Pseudomonas Aeruginosa induce a concentration dependent colorimetric response in Perspex PDA sensors within a time period of 35 to 73 hours. A threshold bacterial concentration of 10̂8-10̂⁹ cells/ml is required to induce colorimetric response.

### Bacterial Enumeration.

Two separate methods were used to calculate the bacterial concentrations at regular intervals during the experiment. Both the methods can reliably measure the bacterial concentrations from the range of 10 cell/ml to 10̂⁷ cells/ml. The calibration curve for the Brewster method was obtained by plotting the growth curves of Pseudomonas Aeruginosa and Staphyloccocus Aureus for initial bacterial concentrations of 10̂2, 10̂3, 10̂4, 10̂5 and 10̂6 cells/ml. When the bacterial concentration is higher than 10̂7 cells/ml the calibration curve and equation are an estimate of the growth trend and the margin for error increases. The method used by Micrim labs to calculate the bacterial concentration is known as plate counting. However if the bacterial concentration is greater than 10̂7 cells/ml the colonies grow in close proximity to each other and individual colonies cannot be identified and counted.

The results obtained for bacterial counts for Plate 3 and Plate 4 from Micrim labs were always lower than the results obtained using the Brewster method (Data present in Appendix 2 and Appendix 3). The lower count obtained could be due to attrition of bacterial cells that occurs during the storage and transport of samples (at 4° C.) from Cirle to Micrim. The discrepancies in the bacterial counts obtained from both methods could also be attributed to the fact that samples for both methods were acquired from different test wells.

It should also be noted that the bacterial counts obtained using the Brewster method provide a more conservative estimation of PDA sensor sensitivity. The results presented were all obtained using the more conservative of the bacterial enumeration methods.

### Glass PDA and Perspex PDA Sensor Incubated in PBS Buffer:

The PDA sensors incubated with Pseudomonas Aeruginosa and Staphylococcus Aureus in PBS did not change color throughout testing. Based on bacterial counts obtained from wells containing Pseudomonas Aeruginosa and Staphylococcus Aureus in PBS we estimate that the lack of nutrients in PBS buffer did not allow the proliferation and survival of bacterial cells beyond 24 hours. The bacteria were not able to reach a threshold concentration (10̂8˜10̂9 cells/ml) which is critical to induce color change in PDA sensors.

### PDA Sensor Response Observed Towards Pseudomonas Aeruginosa and Staphyloccocus Aureus:

In this set of experiments, we noted that the specific PDA formulations created and tested have colorimetric response to Pseudomonas Aeruginosa, but not Staphylococcus Aureus after approximately 4 days of the experiment. This is likely due to several factors, including that, Pseudomonas Aeruginosa quickly form biofilm, whereas Staphylococcus Aureus forms biofilms very slowly. Staphylococcus Aureus is Gram-positive and research had shown that the Lipopolysaccharide, which is the complex glycolipids embedded within the membrane of Gram negative bacteria, is the major component interacting with PDA sensors. We believe that the use of a more porous material to embed the PDA to would allow for enhanced Lipopolysaccharide and biofilm interaction with the PDA. Additionally, changes to the concentration and composition of PDA will improve sensor sensitivity to Staphylococcus. Thus, a sensor can be designed to interact with selected microbes, or to a plurality of microbes.

Following the experiment, the Staphylococcus Aureus cultured in growth media were removed from the plates and all the plates were wrapped with parafilm and alumina foil to store in a 4° C. refrigerator. The plate wells containing PDA sensors were not washed with detergent, so there is still a very small portion of Staphylococcus Aureus left on the PDA sensors. After approximately 30 days, all the glass PDA sensors change color from blue to red in the plate which used to have Staphylococcus Aureus cultured with growth media. The number of Staphylococcus Aureus is hard to estimate since all the bacteria are incubated in 4° C. refrigerator and most of the Staphylococcus Aureus including growth media were removed. A few Perspex PDA sensors also changed color form blue to red in the plate which used to have Staphylococcus Aureus cultured with growth media. The experimental result is estimated since the glass PDA has a faster response time than Perspex PDA. Above observations were conducted with a control group, which did not demonstrate a colorimetric change when stored in the same fashion and temperature conditions.

The experimental results indicate that PDA sensors are also sensitive to Staphylococcus Aureus. However, the response time is very slow as compared to Pseudomonas Aeruginosa and takes up to 30 days probably because Staphylococcus Aureus does not have as much Lipopolysaccharide copies as Gram negative bacteria cell lines do.

### Conclusions

Testing carried out to explore the colorimetric response of circular Perspex and square glass PDA sensors under the influence of bacteria has been completed, reviewed, and summarized. This was an exploratory test without strict acceptance criteria.

The glass PDA sensors displayed a faster response time to Pseudomonas Aeruginosa as compared to the Perspex PDA sensors. We hypothesize that this is due to an inherent difference in the formulation of PDA used to synthesize both the sensor types. The glass PDA sensors were fabricated using a blend of PDA monomers and silica which is conducive towards dip-coating. On the other hand the Perspex PDA sensors were formulated using PDA monomers dissolved in solvents, which is conducive towards spin coating. We hypothesize that the presence of silica micro-domains on the surface of the glass PDA enabled bacterial anchoring, providing better interaction between the bacterial cells and PDA domains.

The PDA sensors have a much faster response time to Pseudomonas Aeruginosa than Staphylococcus Aureus. We hypothesize that this is because Staphylococcus Aureus does not have as many copies of Lipopolysaccharide as Pseudomonas Aeruginosa does due to the inherent difference in these two bacteria strains. Thus, selective or general sensors to microbes can be developed as needed for a particular use.

### Example 2

### Preparation of PDA/Packaging Polymer Materials by Mixing Diacetylene Monomers and Packaging Monomers

In one embodiment, the packaging monomer is prepared by coating a Silicon wafer or glass substrate with (tridecafluoro-1,1,2,2-tetrahydrooctyl)trichlorosilane by keeping the substrate and a drop of the reagent kept in a vial in a desiccator for 30 minutes. First, the base is mixed with the curing agent at a 10:1 ratio by weight. Air bubbles are then removed from the mixture by applying a vacuum and the mixture is poured on the substrate. The resultant silicon or glass monomer is then placed in an oven maintained at 700° C. for 2 hours to make it solidified.

In parallel, PDA is prepared by evaporating 140 ml of diacetylene monomer for at least 4 hours at 60 mbar conditions. 2 mL of DDW (doubly distilled water) is then added to the monomer solution. The mixture is sonicated using intervals for 4 minutes at 70° C. and then cooled to room temperature. The PDA mixture and the silicon or glass polymer are then mixed and cured. Polymerization of PDA is subsequently carried out through exposure of the material to ultraviolet light (254 nm) for several seconds, until it appears blue. In another embodiment, gel is substituted for the silicone or glass polymer.

### Example 3

### Preparation of PDA/Packaging Polymer Materials by Mixing Diacetylene Monomer Vesicles and Packaging Monomers

In some embodiments, diacetylene monomers are dissolved in aqueous solution and small particles/vesicles are constructed. Parameters to be modified are: concentration; pure diacetylene monomers or mixtures with lipids/surfactants/additives to enhance stability; and size of formed particles. Packaging monomers are dissolved in aqueous solution or mild organic solvents (mild—to prevent dissolution of diacetylene particles after mixing). The two solutions are mixed. Parameters to be modified are: ratios; duration before mixing; and degree of polymerization of individual solutions prior to mixing. The mixture is the polymerized. Parameters to be modified are: degree of polymerization; duration; and timing of polymerization (prior or after molding). Molding and curing to desired shapes is then performed.

### Example 4

### Preparation of PDA/Sol-Gel/Packaging Polymer Materials by Mixing Diacetylene Monomors, Silica Precursors and Packaging Monomers

In one embodiment, the packaging monomer is prepared by coating a Silicon wafer or glass substrate with (tridecafluoro-1,1,2,2-tetrahydrooctyl)trichlorosilane by keeping the substrate and a drop of the reagent kept in a vial in a desiccator for 30 minutes. First, the base is mixed with the curing agent at a 10:1 ratio by weight. Air bubbles are then removed from the mixture by applying a vacuum and the mixture is poured on the substrate. The resultant silicon or glass monomer is then placed in an oven maintained at 700° C. for 2 hours to make it solidified.

The sol-gel component is prepared by mixing tetramethoxysilane (TMOS), water and 0.62M HCL (4.41:2.16:0.06 v:v:v). The mixture is incubated for one hour with stirring at 4° C., diluted with water 1:1 (v:v) and then evaporated for approximately six minutes at 60 mbar. Then, after sonication in water, lipid/polydiacetylene (PDA) vesicles (PDA/DMPC 3:2, mole ratio) were prepared by dissolving the lipid components in chloroform/ethanol and drying together in vacuo. Vesicles were subsequently prepared in DDW by probe-sonication of the aqueous mixture at 70° C. for 3 min. The vesicle solution was then cooled at room temperature for an hour and kept at 4° C. overnight. 7 mM DMPC/PDA liposomes are diluted with Tris pH 7.5 1:1 (v:v). The solution of liposomes and the solution of silica gel are mixed 1:1 (v:v) and immediately placed in a 384-well ELISA plates (15 μl in each well). Gelation then occurs for 30 minutes at room temperature. After gelation, each well is filled with a Tris pH 7.5 solution for storing in a refrigerator. After a minimum of overnight in the refrigerator, the mixture is polymerized for 2 minutes before it is heated to room temperature (30 minutes).

The PDA/sol-gel mixture is then prepared as follows. 140 microliters of diacetylene/dimyristoylphosphatidylcholine (DMPC) total concentration 7 mM, mole ratio 3:2 (PDA:DMPC) is evaporated for at least 4 hours at 60 mbar conditions. 2 mL of DDW is then added to the diacetylene/DMCP solution and sonicated for 6 minutes (3 minutes with heat). After cooling to room temperature, the diacetylene/DMCP solution is mixed with the pre-solidified sol-gel component. The mixture is allowed to solidify and PDA is polymerized using ultraviolet irradiation at 254 nm. Packaging monomers are added to the mixture prior to PDA polymerization.

### Example 5

### Preparation of PDA/Silica Materials to be Coated onto Packaging Materials

Precursor solutions were synthesized from tetraethyl orthosilicate (TEOS), diacetylene (TRCDA, or 10,12-tricosadiynoic acid) and HNO₃ catalyst prepared in a tetrahydrofuran (THF)/water solvent at room temperature. The final reactant mole ratios were 1:9:312:0.13:0.05 (TRCDA:TEOS:THF:HNO₃:H₂O). After one day aging at ambient temperature, the silica/PDA sol solution was filtered through 0.45 μm nylon and kept at −200° C.

For deposition on a packaging material, the material to be coated was dipped in the silica/PDA sol and kept immersed for 1 minute. After this, the packaging material was pulled out at withdrawal speed of approximately 35 mm/s. Following air-drying, uniform thin films are ultraviolet-irradiated (254 nm) for 1 minute to produce the blue-phase PDA thin film material.

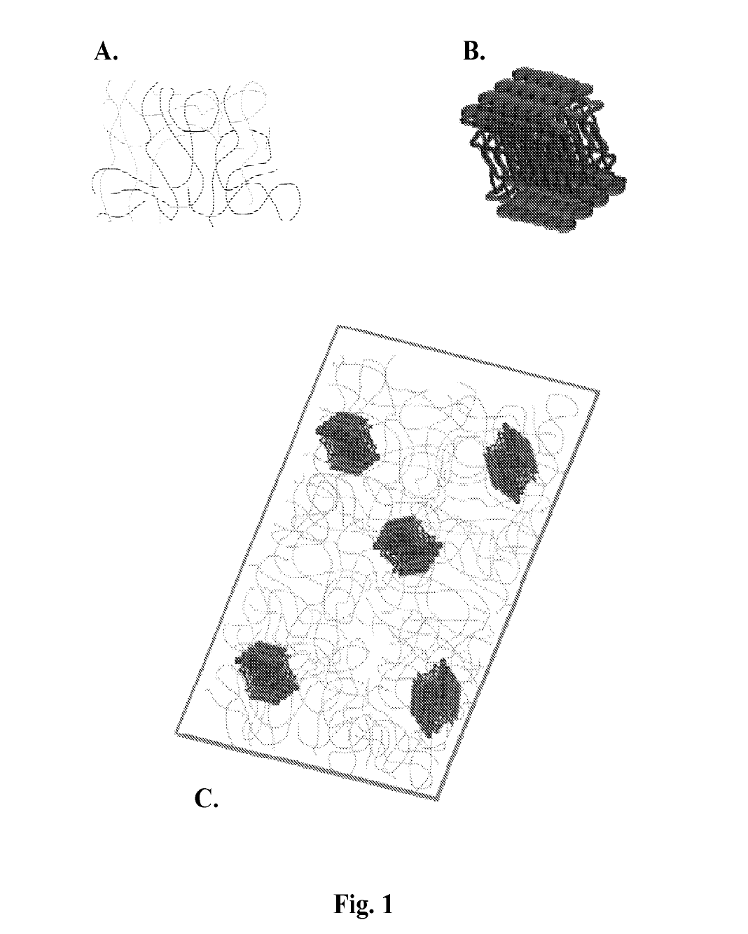

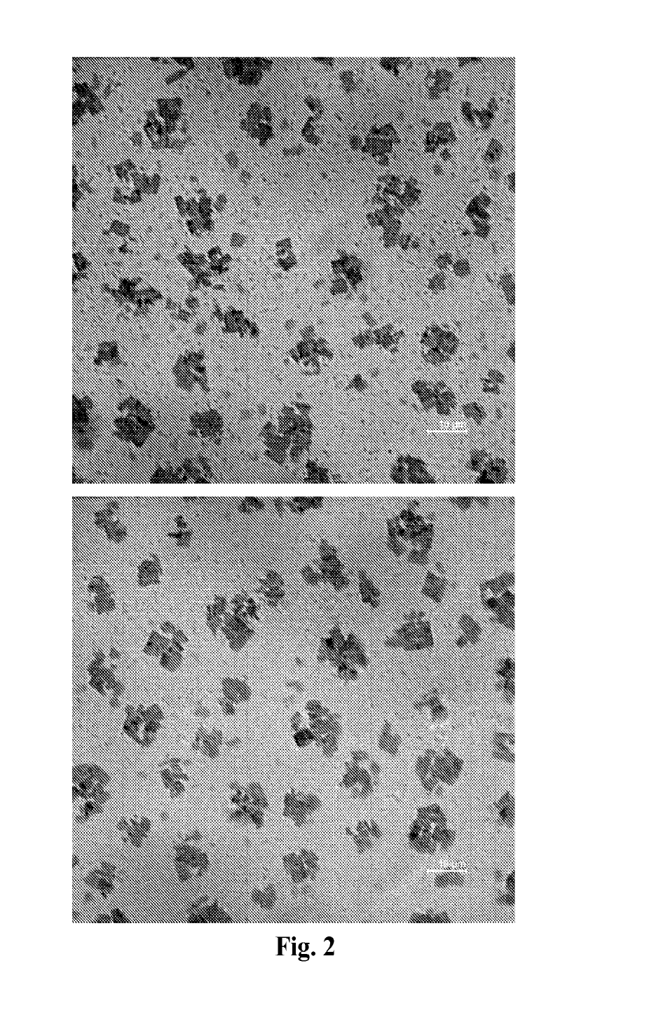

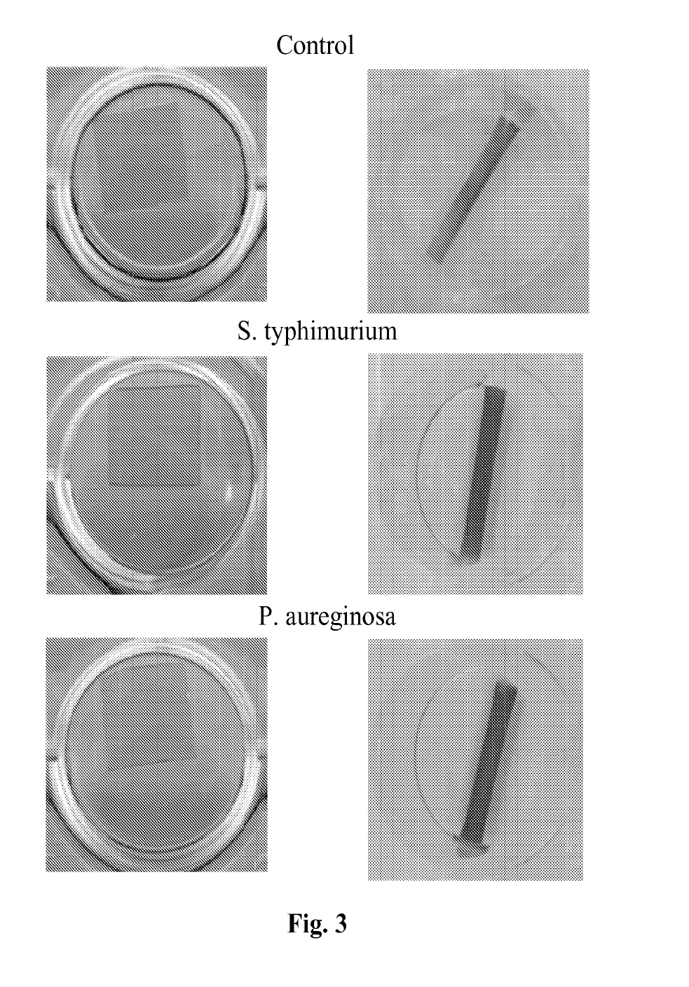

FIG. 1 shows a schematic of discrete diacetylene lamellar domains distributed across a sol-gel, packaging polymer, or sol-gel/packaging polymer surfaces. FIG. 2 shows microscopy images of discrete diacetylene lamellar domains distributed across a sol-gel surface. FIG. 3 shows the results of patches and tubing coated with the sol-gel/PDA solutions, which patches and tubing were subsequently contacted with either a control, S. typhimurium or P. aureginosa. This figure demonstrates that PDA solutions comprising silica can be coated onto packaging materials and used to detect microbes and/or microbial products.

### Example 6

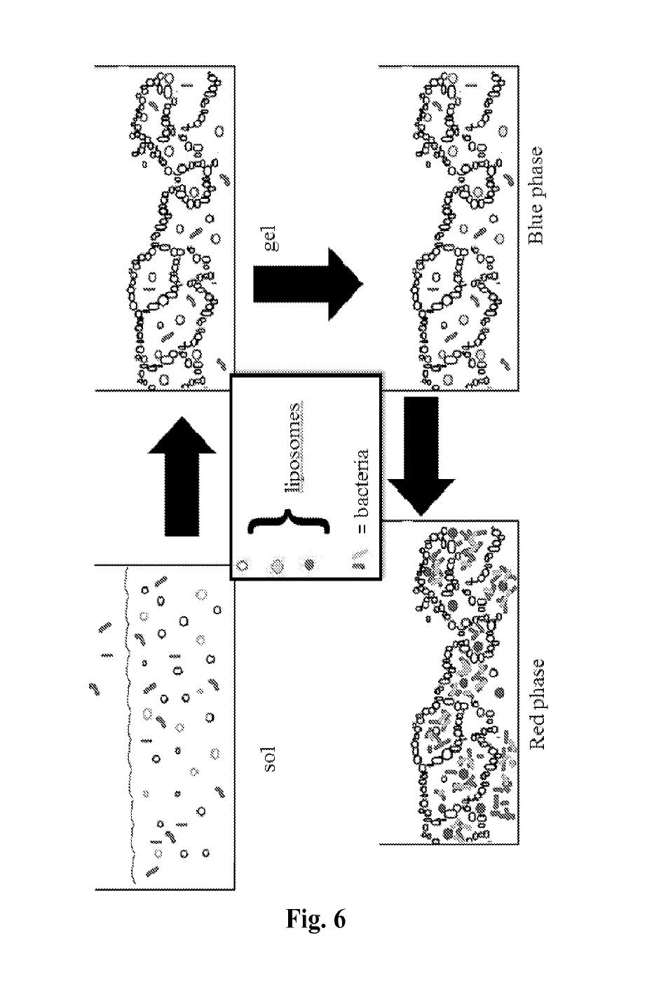

### Preparation of PDA/Vesicle Materials to be Attached to Packaging Materials (See, FIG. 6)

The synthesis of PDA films will be done using a two-step procedure described by Silbert et al. [Silbert L, Shlush I B, Israel E, Porgador A, Kolusheva S, Jelinek R. 2006. Applied and Environmental Microbiology. 72: 7339-7344]. The first-step comprises of creating vesicles using PDA monomers. These vesicles are then trapped to agar gels, before polymerizing the entire construct. More specifically, vesicles containing DMPC and 10,12-tricosadiynoic acid (2:3 molar ratio) will be prepared at a concentration of 1 mM. The lipids will then be dried together in vacuo. Following evaporation, distilled water will be added and the suspension will then be probe sonicated at 70° C. The resultant vesicle solution will be cooled at 4° C. overnight and then polymerized by irradiation at 254 nm for 0.5 minutes.

A chromatic lipid-PDA agar matrix is then prepared as follows. Unpolymerized PDA vesicles at a concentration of 5 mM will be added right after the sonication stage to hot LB agar. The mixture will then be cooled to room temperature. After solidification of the agar, the plate is kept at 4° C. for 2 days and polymerized by irradiation (254 nm, 40 s) in a UV cross-linker (UV-8000; Stratagene, California).

Four different types of bacteria namely Serratia spp (gram −ve), Pseudomonas (gram −ve), Staphylococcus aureus (gram +ve), and Staphylococcus pneumoniae (gram +ve) and fusarium (fungi) which are commonly associated to keratitis are used to challenge the PDA film sensors. Different concentrations of bacteria/fungi are spiked into the lens solutions to determine the detection limit and detection range. More specifically, bacterial samples are purchased from America Type Culture Collection (ATCC) and cultured as per provider specifications. A mounted digital camera is used to acquire images of PDA films in the presence of different concentrations of bacteria/fungi every 30 minutes for a period of 10 hours. Images are evaluated to calculate the sensor response time to bacteria/fungal contamination. The minimum detection capabilities of the film is also evaluated.

The PDA/vesicle films are further evaluated for stability in multipurpose contact lens solution at different temperature and pH. More specifically, PDA films are stored in the contact lens solution for a period 60 days. The films are also exposed to temperature and pH fluctuations. The PDA film storage lens solutions is then compared to normal lens solutions using mass spectroscopy to determine any constitutional changes which would indicate film leeching or degradation. In order to determine the stability of the PDA films mass spectroscopy is used to evaluate and obtain the chemical signatures of contact lens solutions. The chemical signatures of the bottled solution are compared with the signature obtained from the PVA film storage solution to detect PDA or agar leeching/degradation. The films are subjected to high temperature and pH fluctuations to evaluate their stability.

### Example 7

### Glass Slides Coated with PDA Solgel Films (Dip-Coating)

1. Precursor molar ratios for the dip-coating solution are about: 1:9:312:0.13:40 (diacetylene monomers (TRCDA):silica precursor (TEOS):THF (organic solvent):HNO₃:H₂O). Precursor solution has to be prepared at least 24 h before the experiment in order to complete multiple hydrolysis reactions of silica precursor molecules (TEOS). A dip-coating solution preparation is a two-step process. First, TRCDA solution (A) will be prepared from diacetylene monomers dissolved in a THF solvent (45 mg/ml). TEOS solution (B) will be prepared separately by a mixing of TEOS with THF and the nitric acid aqueous solution (0.15 N) at the volume ratios of 1:5:0.25 correspondingly. 0.15 N nitric must be prepared with a double deionized water separately. Then, B solution will be stirred for an hour using vortex mixer following by a 24 hour storage in the incubator at the 30° C. Right before the dipping, A and B solutions will be mixed together for an hour using vortex mixing in order to get a homogeneous solution.
2. Glass surface was cleaned and activated by incubation in methanol for 10 minutes. The glass was removed from methanol solution and allowed to air dry for 30 minutes. The PDA was coated onto glass surfaces using a dip-coating technique.
3. The withdrawal speed of the dip coating equipment was controlled at 35 mm/min, T=20° C.
4. The resulting coatings were air-dried for a period of 8 hours, and then polymerized with UV light (254 nm) (1 min each side). This is an embodiment of the structure described herein.
   Glass Slides Spin-Coated with PDA Films
1. Precursor molar ratios for the dip-coating solution are: 1:9:312:0.13:40 (diacetylene monomers (TRCDA):silica precursor (TEOS):THF (organic solvent):HNO₃:H₂O). Precursor solution has to be prepared at least 24 h before the experiment in order to complete multiple hydrolysis reactions of silica precursor molecules (TEOS). A dip-coating solution preparation is a two-step process. First, TRCDA solution (A) will be prepared from diacetylene monomers dissolved in a THF solvent (45 mg/ml). TEOS solution (B) will be prepared separately by a mixing of TEOS with THF and the nitric acid aqueous solution (0.15 N) at the volume ratios of 1:5:0.25 correspondingly. 0.15 N nitric must be prepared with a double deionized water separately. Then, B solution will be stirred for an hour using vortex mixer following by a 24 hour storage in the incubator at the 30° C. Right before the dipping, A and B solutions will be mixed together for an hour using vortex mixing in order to get a homogeneous solution.
2. Precursor was coated onto glass slides using spin coating techniques. Spin coating was conducted using Laurell WS-650 Mz-23NPP Single Wafer Spin Processor at 2000 rpm for 30 seconds.
3. The resulting coatings were air-dried for a period of 8 hours, and then polymerized with UV light (254 nm) (1 min each side). This is an embodiment of the structure described herein.
   PMMA Slides Coated with PDA Films Containing Silica/ZnO Microparticles
1. Precursor molar ratios for the dip-coating solution are: 1:9:312:0.13:40 (diacetylene monomers (TRCDA):silica precursor (TEOS):THF (organic solvent):

HNO₃:H₂O). Precursor solution has to be prepared at least 24 h before the experiment in order to complete multiple hydrolysis reactions of silica precursor molecules (TEOS). A dip-coating solution preparation is a two-step process. First, TRCDA solution (A) will be prepared from diacetylene monomers dissolved in a THF solvent (45 mg/ml). TEOS solution (B) will be prepared separately by a mixing of TEOS with THF and the nitric acid aqueous solution (0.15 N) at the volume ratios of 1:5:0.25 correspondingly. 0.15 N nitric must be prepared with a double deionized water separately. Then, B solution will be stirred for an hour using vortex mixer following by a 24 hour storage in the incubator at the 30° C. Right before the dipping, A and B solutions will be mixed together for an hour using vortex mixing in order to get a homogeneous solution.
2. Precursor was coated onto PMMA slides using spin coating techniques. Spin coating was conducted using Laurell WS-650 Mz-23NPP Single Wafer Spin Processor at 2000 rpm for 30 seconds.
3. The resulting coatings were air-dried for a period of 8 hours, and then polymerized with UV light (254 nm) (1 min each side). This is an embodiment of the structure described herein.
   PMMA Transparent Slides Coated with PDA (Dip-Coating)

The PDA was coated onto PMMA substrates using a dip-coating technique. Poly(methyl methacrylate) (PMMA) transparent lightweight plastic slides (24 mm*60 mm*1 mm) were used as a substrates, while the dip-coating solution consisted of TRCDA (diacetylene monomers) in THF (tetra hydrofuran) organic solvent (35 mg/ml). The resulting coatings were air-dried for a period of 8 hours, and then polymerized with UV light (254 nm) (1 min each side). This is an embodiment of the structure described herein.

PMMA Transparent Slides Coated with PDA (Spin-Coating)

The spin-coating solution (PDA precursor solution) consists of TRCDA dissolved in Tetra hydrofuran (THF):Methylene Chloride (DCM) (1:1) solution with a final diacetylene concentration of 40 mg/ml in it. Diacetylene monomer is a hydrophobic molecule that dissolves easily either in both solvents separately or in their mixture. TRCDA dissolves without a vortex mixing, but still, it is better to use vortex in order to achieve a totally homogeneous solution. This solution has to be filtered to remove aggregates before each usage. For that purpose we use Nylon membrane filter with a pore size of 0.45 μm. The filtration is conducted using a manually held syringe.

Precursor was coated onto PMMA slides using spin coating techniques. Spin coating was conducted using Laurell WS-650 Mz-23NPP Single Wafer Spin Processor at 2000 rpm for 30 seconds.

The resulting coatings were air-dried for a period of 8 hours, and then polymerized with UV light (254 nm) (1 min each side). This is an embodiment of the structure described herein.

### PMMA/PDA Hybrid Polymer

a) PDA monomers can be embedded into PMMA polymer matrices to create a flexible plastic PDA sensor.
b) The fabrication method used to create these sensors is referred to as CASTING. In order to cast a layer of PMMA/PDA layer in multi-well polypropelene plate, commercial PMMA powder (average Mw≈120,000) is to be dissolved with TRCDA (diacetylene monomer) powder in methylene chloride (organic solvent). Then, the solution must be added to the molds (plate) for the drying. The resulted layers are to be polymerized with 254 nm UV light. This is an embodiment of the structure described herein.

### Porous Sol-Gel/PDA Matrix

1) Creation of sol-gel that maintain Liposomes and buffer
a. Lipids solution of DMPC and TRCDA were mixed using a vortex mixer for 5 minutes and the solvents were evaporated using a rotary evaporator.
b. 2 ml DDW was added to the components left behind after complete evaporation of solvents and sonication was carried out for 2 minutes to enable formation of liposomes.
c. The liposomes were allowed to rest for ˜8 hours or overnight at 4° C.
d. Sol gel was created according to the following procedure:
   Mix gel compounds long chain monomer TMOS:water: 0.62M HCL (:1.32:3.09:2.16:0.06 v:v:v). (the long chain monomer is XDV 554 BS-15PEG ICGD-CNOS)
   Incubation 1 h with stirrer in 4° C.
   Dilution with water 1:1 (v:v) and evaporation ˜6 min 60 mbar.
   7 mM liposomes after sonication in water (regular protocol for preparation) were dilute with Tris pH 7.0 1:1 (v:v).
   Solution of liposomes and solution of silica gel (after evaporation) were mixing 1:1 (v:v) and immediately placed in a multiwell plate. 200 ul in each well.
   Gelation was for ˜30 min at room temperature
   After gelation cover each well with DDW for storing in refrigerator for
   ˜8ₕ or overnight.

2) After overnight at 4 C, polymerization of the liposomes
a. The ELISA plates with sol gel got to the room temperature.
b. Polymerization was conducted using 254 nm of UV irradiation light for 60 seconds.

### Coating PDA Nanotubes on Glass Substrates Using Dip-Coating

1) Glass coverslips were treated with Piranha solution (1:2, hydrogen peroxide:sulfuric acid) for 15 minutes at 120° C. to create a negative surface charge.

2) Coverslips were then washed using DI water 3× for 5 minutes with shaking.
3) Coverslips were incubated in 10% by volume HCl solution for 10 minutes at 90° C.
4) Coverslips were then washed using DI water 3× for 5 minutes with shaking.
5) Coverslips were incubated in 1% by weight NaOH solution for 10 minutes at room temperature.
6) Coverslips were then washed using DI water 3× for 5 minutes with shaking.
7) The coverslips were dried in vacuum for 30 minutes.
8) PDA NTs solution of 1 mg/ml was made in hexane using ultra-sonication for 10 minutes.
9) Each charged glass coverslip was added to the PDA NT solution and sonicated for 2 minutes.
10) The positively charged PDA NTs will coat the negatively charged glass surfaces via ionic interaction.
11) The coverslips was removed from the solution and rinsed in 50 ml hexane to remove any unbound or loosely bound nanotubes.
12) The coverslips were be dried in vacuum at room temperature for 10 hours. This is an embodiment of the structure described herein.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to the measuring technique and the numerical value. In addition, the phrase “about ‘x’ to ‘y’” includes “about ‘x’ to about ‘y’”.

While only a few embodiments of the present disclosure have been shown and described herein, it will become apparent to those skilled in the art that various modifications and changes can be made in the present disclosure without departing from the spirit and scope of the present disclosure. All such modification and changes coming within the scope of the appended claims are intended to be carried out thereby.

1. A container for detection of a microbe or microbial product in a fluid comprising:
a polydiacetylene (PDA) material, wherein the microbe or microbial product contacts the PDA material and a change of PDA material color indicates detection of the microbe or microbial product, wherein the PDA material is disposed on a structure or on a portion of the container.

2. The container of claim 1, wherein the PDA material is selected from the group consisting of: a PDA layer, a PDA nanoparticle, a PDA vesicle, and a PDA micelle.

3.-4. (canceled)

5. The container of claim 2, wherein the PDA material is conjugated with a capturing agent, wherein the capturing agent binds to the microbe or microbial product.

6. (canceled)

7. The container of claim 2, wherein a charged material is attached to a portion of the PDA surface, wherein the charged material binds to an oppositely charged microbe or microbial product.

8. The container of claim 2, wherein a capturing agent is disposed on a surface of the structure or a portion of the container, adjacent the PDA material, wherein the capturing agent binds to the microbe or microbial product.

9. The container of claim 2, wherein a charged material disposed on a surface of the structure or a portion of the container, adjacent the PDA material, wherein the charged material binds to an oppositely charged microbe or microbial product.

10.-17. (canceled)

18. The container of claim 2, wherein the structure is a filter that includes the PDA material.

19. The container of claim 1, wherein the structure is a spherical object placed inside the container and includes the PDA material.

20. (canceled)

21. The container of claim 19, wherein the spherical object floats in the fluid present in the container.

22.-23. (canceled)

24. The container of claim 1, further comprising a light source in a cap of the container, wherein the PDA material is a fluorescent PDA material.

25. The container of claim 1, further comprising a cap, wherein the inner surface of the cap has the PDA material disposed thereon.

26.-27. (canceled)

28. The container of claim 1, further comprising a tab that when removed exposes the fluid in the container to the PDA material.

29. The container of claim 1, further comprising a secondary compartment, wherein the PDA material is present in the secondary compartment, wherein the fluid in the container only comes into contact with the PDA material after the container after an event occurs prior to opening the container for the first time after being sealed by the manufacturer.

30. The container of claim 29, wherein the event includes turning a cap to open the container.

31. (canceled)

32. The container of claim 1, further comprising a secondary compartment and a tertiary compartment, wherein the PDA material is present in the secondary compartment, wherein an activating solution in the tertiary compartment, wherein a boundary between the secondary compartment and the tertiary compartment is made of a material that is dissolved by the microbe or microbial product, wherein if a microbe or microbial product is present, the microbe or microbial product dissolves the boundary and causes the PDA material and the activating material to come into contact with one another causing the PDA to change color.

33. (canceled)

34. A biosensor comprising:
a polydiacetylene (PDA) material, wherein a microbe or microbial product in a fluid contacts the PDA material and a change of PDA material color indicates detection of the microbe or microbial product, wherein the PDA material is disposed on a structure of the biosensor or the biosensor.

35.-53. (canceled)

54. The biosensor of claim 52, wherein the spherical object floats in the fluid present in the container.

55.-61. (canceled)

62. The biosensor of claim 54, further comprising a secondary compartment, wherein the PDA material is present in the secondary compartment, wherein the fluid in the container only comes into contact with the PDA material after the container after an event occurs prior to opening the container for the first time after being sealed by the manufacturer.

63.-64. (canceled)

65. The biosensor of claim 34, further comprising a secondary compartment and a tertiary compartment, wherein the PDA material is present in the secondary compartment, wherein an activating solution in the tertiary compartment, wherein a boundary between the secondary compartment and the tertiary compartment is made of a material that is dissolved by the microbe or microbial product, wherein if a microbe or microbial product is present, the microbe or microbial product dissolves the boundary and causes the PDA material and the activating material to come into contact with one another causing the PDA to change color.

66.-70. (canceled)

71. The biosensor of claim 34, wherein the PDA material is a PDA softgel, wherein the PDA softgel is made from a precursor solution of: about 1:9:312:0.13:40 (diacetylene monomers (TRCDA):silica precursor (TEOS):THF (organic solvent):HNO3:H2O).

72.-84. (canceled)
